(19) United States
(12) Patent Application Publication
(10) Pub. No.: US 2014/0288014 A1
(43) Pub. Date: Sep. 25, 2014

(54) COMPOSITIONS OF CRUDE ALGAL OIL

(71) Applicant: Aurora Algae, Inc., Hayward, CA (US)

(72) Inventors: Bertrand Vick, Oakland, CA (US); Andrew Thompson, Oakland, CA (US); Michelle L. Collins, Oakland, CA (US); Jeffrey Gerard Hippler, South Charleston, WV (US); James Astwood, Alameda, CA (US)

(21) Appl. No.: 14/218,153

(22) Filed: Mar. 18, 2014

Related U.S. Application Data
(60) Provisional application No. 61/800,114, filed on Mar. 15, 2013.

(51) Int. Cl. A61K 31/202; A61K 31/7032

(52) U.S. Cl. CPC A61K 31/202; A61K 31/7032
USPC 514/25; 514/560

(57) ABSTRACT

The present invention provides compositions for a crude algal oil and methods of making thereof. The compositions can be used to produce a highly pure omega-3 eicosapentaenoic acid (EPA) formulation.

### CROSS-REFERENCES TO RELATED APPLICATIONS

This application claims priority to U.S. Provisional Application No. 61/800,114, filed Mar. 15, 2013, the contents of which are hereby incorporated by reference in the entirety for all purposes.

### BACKGROUND OF THE INVENTION

Algae, e.g., microalgae, are photosynthetic organisms that convert light energy and carbon dioxide into biomass including lipids, carbohydrates, and proteins. Marine algae strains can produce a high percentage of total lipids (up to 30-70% of dry weight) (Ward O P and Singh A, Process Biochem, 2005, 40(12):3627-3652). Various algal strains can produce omega-3 polyunsaturated fatty acids, e.g., eicosapentaenoic acid (EPA; C20:5n-3) and docosahexaenoic acid (DHA; C22:6-n3). For example, algae of the genus Nannochloropsis are abundant in EPA as well as the omega-7 fatty acid, palmitoleic acid (C16:1n-7), and the omega-6 fatty acid, arachidonic acid (ARA;C20:4n-6) Algae is becoming an increasingly important source of nutritionally important omega-3 polyunsaturated fatty acids.

The composition of the fatty acids varies among the different algal strains. For instance, it has been reported that Nannochloropsis sp. contains 26.7% (by weight from total fatty acids) EPA and DHA (Hu H and Gao K, Biotechnol Lett, 2003, 25(5):421-425), while Nannochloropsis oceania has 23.4% EPA (Patil et al., Aquac Int, 2007, 15(1):1-9) and Nannochloropsis salina contains about 28% EPA (Van Wagenen et al., Energies, 2012, 5(3):731-740).

The consumption of omega-3 fatty acids provides various health benefits such as to prevent cardiovascular disease, enhance brain function, and diminish symptoms of inflammatory conditions such as rheumatoid arthritis, Crohn's disease, and ulcerative colitis. For instance, the EPA-only therapeutic formulation Vascepa® is approved for the treatment of hypertriglyceridemia. Because omega-3 fatty acids are not synthesized de novo in the human body, these fatty acids must be derived from agricultural and aquacultural sources. There is a growing demand for algal-derived omega-3 EPA-only compositions in the nutraceutical and pharmaceutical industry. The present invention addresses this and other related needs by providing a novel composition of crude algal oil, as well as related fatty acid compositions produced from the algal oil and methods for making and using these compositions.

### BRIEF SUMMARY OF THE INVENTION

The invention relates to compositions of a crude algal oil that is useful for producing nutraceutical and pharmaceutical compounds.

Thus, in one aspect, the present invention provides a crude algal oil composition. The composition may include about 30% to about 35% eicosapentaenoic acid (EPA), less than about 10% arachidonic acid (ARA), and less than about 0.5% docosahexaenoic acid (DHA) by weight of total fatty acids. In some embodiments, DHA of the crude algal oil is less than about 0.1% by weight of total fatty acids. In other embodiments, the composition is substantially free of DHA.

In another aspect, the present invention provides a crude algal oil composition that includes a triacylglycerol fraction of at least about 2% by weight of the total fatty acids, wherein about 7% of the eicosapentaenoic acid (EPA) of the crude algal oil is in the triacylglycerol fraction, wherein about 11% of the arachidonic acid (ARA) of the crude algal oil is in the triacylglycerol fraction, and wherein substantially no docosahexaenoic acid (DHA) is in the triacylglycerol fraction.

In yet another aspect, the present invention provides a crude algal oil composition that includes a monogalactosyldiacylglycerol (MGDG) fraction of at least about 5% by weight of the total fatty acids, wherein about 37% of the eicosapentaenoic acid (EPA) of the crude algal oil is in the MGDG fraction, wherein about 14% of the arachidonic acid (ARA) of the crude algal oil is in the MGDG fraction, and wherein substantially no docosahexaenoic acid (DHA) is in the MGDG fraction.

The EPA to ARA (EPA:ARA) ratio of the crude oil composition may range from about 5:1 to about 40:1. Alternatively, the EPA:ARA ratio ranges from about 6:1 to about 30:1.

The crude algal oil contains various lipids fractions such as phospholipids (e.g., phosphatidylinositols (PIs), phosphatidylserines (PSs), phosphatidylcholines (PCs), phosphatidylglycerols (PG), and phosphatidylethanolamines (PE)); galactolipids (e.g., digalactosyldiacylglycerols (DGDGs) and monogalactosyldiacylglycerols (MGDGs)); neutral glycolipids (e.g., monoacylglycerols (MAGs), diacylglycerols (DAGs), and triacylglycerols (TAGs)); free fatty acids (FFA), sterol esters (e.g., cholesterol esters); polar glycolipids (e.g., sulfoquinovosyldiacylglycerols (SQDGs)); and diacylglycerol trimethylhomoserines (DGTSs).

In some embodiments, 80% of the EPA in the crude algal oil is in polar lipids and about 20% of the EPA is in neutral lipids. In some embodiments, about 37% of the EPA in the crude algal oil is in monogalactosyldiacylglycerol lipids. In some embodiments, about 10% of the EPA in the crude algal oil is in free fatty acids, phosphatidylglycerol lipids or digalactosyldiacylglycerol lipids.

The crude algal oil may be derived from an algal biomass, for example, after extraction by an organic solvent such as ethanol. The algal biomass may include Nannochloropsis. In some embodiments, the Nannochloropsis is selected from the group consisting of Nannochloropsis gaditana, Nannochloropsis granulate, Nannochloropsis limnetica, Nannochloropsis oceanica, Nannochloropsis oculata and Nannochloropsis salina.

In some embodiments, the biomass may be a wet biomass. In preferred embodiments, the algal biomass is a dried algal biomass. The dried algal biomass may include at least about 10% lipids, at least about 15% carbohydrates, at least about 25% protein, at least about 3% moisture, and at least about 1% ash. The Nannochloropsis may be a mutant strain, for example, the T661 strain, as described in U.S. Provisional Patent Application Nos. 61/800,029 and 61/800,114, both filed on Mar. 15, 2013.

In some embodiments, the crude algal oil is further processed to produce an omega-3 composition comprising about 60-99% EPA by weight. In some cases, the EPA contents in such an omega-3 composition may be as high as 90-99% by weight.

Also provided herein are methods for making the crude algal oil composition described above. The method includes (a) obtaining whole algal biomass; (b) adding an organic solvent such as an alcohol (e.g., ethanol) to the algal biomass; and (c) extracting liquid from the algal biomass, thereby obtaining the crude algal oil. In some embodiments of the method, the algal biomass is a wet algal biomass. In the alternative, the algal biomass is a dried algal biomass. The dried algal biomass may include at least about 10% lipids, at least about 15% carbohydrates, at least about 25% protein, at least about 3% moisture, and at least about 1% ash. The algal biomass may include Nannochloropsis cells. In some embodiments, Nannochloropsis is selected from the group consisting of Nannochloropsis gaditana, Nannochloropsis granulate, Nannochloropsis limnetica, Nannochloropsis oceanica, Nannochloropsis oculata and Nannochloropsis salina. The Nannochloropsis may be a mutant strain, such as the T661 strain described in U.S. Provisional Patent Application No. 61/800,029. In some embodiments, the crude algal oil produced according to the method described herein has less than about 0.1% DHA by weight of total fatty acids. The crude algal oil may be substantially free of DHA. In some embodiments, the EPA:ARA weight ratio may range from about 5:1 to about 40:1. Optionally, the EPA:ARA weight ratio ranges from about 6:1 to about 30:1.

In some embodiments, about 80% of the EPA in the crude algal oil is in polar lipids and about 20% of the EPA is in neutral lipids. In some embodiments, about 37% of the EPA in the crude algal oil is in monogalactosyldiacylglycerol lipids. In some embodiments, about 10% of the EPA in the crude algal oil is in free fatty acids, phosphatidylglycerol lipids or digalactosyldiacylglycerol lipids.

Other objects, features, and advantages of the present invention will be apparent to one of skill in the art from the following detailed description and figures.

### BRIEF DESCRIPTION OF THE DRAWINGS

FIG. 1 shows the percentage of fatty acid (e.g., EPA, ARA and DHA) in one example of crude algal oil derived from four Nannochloropsis strains.

FIG. 2 illustrates the EPA to ARA (EPA:ARA) ratio in one example of crude algal oil prepared from four Nannochloropsis strains.

FIG. 3 illustrates the fatty acid distribution in the polar lipids and neutral lipids of one example of crude algal oil.

FIG. 4 illustrates the EPA and ARA distributions in the lipid fractions of one example of crude algal oil, in particular, the monoacylglycerol (MAG), diacylglycerol (DAG), triacylglycerol (TAG), free fatty acid (FFA), cholesterol ester/hydrocarbon (CE/HC), phosphatidylcholine (PC), phosphatidylglycerol (PG), phosphatidylethanolamine (PE), phosphatidylinositol (PI), phosphatidylserine (PS), sulfoquinovosyldiacylglycerol (SQDG), digalactosyldiacylglycerol (DGDG), diacylglycerol trimethylhomoserine (DGTS), monogalactosyldiacylglycerol (MGDG) fractions.

FIG. 5 illustrates the distribution of fatty acids in the polar lipids, such as the phospholipids, diacylglycerol trimethylhomoserine (DGTS), monogalactosyldiacylglycerol (MGDG), digalactosyl diacylglycerol (DGDG), and sulfoquinovosyldiacylglycerol (SQDG) in one exemplary embodiment of the crude algal oil described herein.

### DETAILED DESCRIPTION OF THE INVENTION

### I. INTRODUCTION

The inventors have discovered novel compositions of a crude algal oil that contains high levels of EPA, low levels of ARA, and substantially no DHA. Provided herein are also methods for making and using the crude oil.

### II. DEFINITIONS

The term “crude algal oil” refers to an oil composition that is directly extracted from an algal biomass without further processing. It is the resulting oil-based liquid product produced from the extraction of biomass with an organic solvent such as an alcohol, e.g., ethanol. The crude algal oil can contain free fatty acids, lipids comprising fatty acids, waxes and polar solubles. The fatty acids are distributed in neutral lipids and polar lipids, e.g., monoacylglycerol (MAG), diacylglycerol (DAG), triacylglycerol (TAG), free fatty acid (FFA), phosphatidylcholine (PC), phosphatidylglycerol (PG), phosphatidylethanolamine (PE), sulfoquinovosyldiacylglycerol (SQDG), digalactosyldiacylglycerol (DGDG), diacylglycerol trimethylhomoserine (DGTS), and monogalactosyldiacylglycerol (MGDG).

The term “refined oil” refers to an oil that is obtained from a crude algal oil after it has undergone the standard processing, such as refining, bleaching, and/or deodorizing.

The term “alga” refers to a marine algal cell or alga. For instance, the term “algae” encompasses macroalgae and microalgae. Preferably, the algae in the present invention are selected from the genus Nannochloropsis. In more preferred embodiments, the Nannochloropsis is of the species Nannochloropsis gaditana, Nannochloropsis granulate, Nannochloropsis limnetica, Nannochloropsis oceanica, Nannochloropsis oculata or Nannochloropsis salina.

The term “lipid” includes phospholipids, free fatty acids, esters of fatty acids, triacylglycerols, diacylglycerols, monoacylglycerols, lysophospholipids, soaps, phosphatides, sterols and sterol esters, carotenoids, xanthophylls (e.g., oxycarotenoids), hydrocarbons, and other lipids known to one of ordinary skill in the art.

The term “neutral lipid” includes triacylglycerols, diacylglycerols, monoacylglycerols, free fatty acids, sterol esters, etc.

The term “polar lipid” includes phospholipids, such as phosphatidylinositol, phosphatidylserine, phosphatidylcholine, phosphatidylglycerol and phosphatidylethanolamine, polar glycolipids, galactolipids, and the like.

The term “eicosapentaenoic acid” or “EPA” refers an omega-3 polyunsaturated fatty acid (denoted as C20:5-n3) and any moiety to which it is attached. It is a carboxylic acid with a 20-carbon chain and five cis double bonds; the first double bond is located at the third carbon from the omega end.

The term “docosahexaenoic acid” or “DHA” refers an omega-3 polyunsaturated fatty acid (denoted as C22:6-n3) and any moiety to which it is attached. It is a carboxylic acid with a 22-carbon chain and six cis double bonds; the first double bond is located at the third carbon from the omega end.

The term “arachidonic acid” or “ARA” refers an omega-6 polyunsaturated fatty acid (denoted as C20:4-n6) and any moiety to which it is attached. It is a carboxylic acid with a 20-carbon chain and four cis-double bonds; the first double bond is located at the sixth carbon from the omega end.

The term “biphasic extract” refers to an extract comprising at least two phases, such as an aqueous phase and an oil phase.

The term “about” when used in connection with a definitive value refers to a variation of 10% more or less than the value recited. For instance, the term “about 50%” defines a range of 50±5%.

Whenever reference is made herein to a fatty acid concentration (percentage), unless otherwise indicated, said concentration (percentage) is calculated by weight of the total amount of fatty acids.

As used herein, the following terms have the meanings ascribed to them unless specified otherwise.

### III. DETAILED DESCRIPTION OF EMBODIMENTS

A. Crude Algal Oil Composition

The inventors have advantageously discovered a crude algal composition that contains about 30% to about 35% EPA (by weight of total fatty acids), e.g., about 30%, 30.5%, 31%, 31.5, 32%, 32.5%, 33%, 33.5%, 34.5%, 35%, 35.5% EPA, less than 10% ARA(by weight of total fatty acids), e.g., 0.5%, 1%, 2%, 3%, 4%, 5%, 6%, 7%, 8%, 9% or less than 10% ARA, and less than 0.5% DHA (by weight of total fatty acids), e.g., 0%, 0.1%, 0.2%, 0.3%, 0.4% or less than 5% DHA in weight by total fatty acid content. The percentage of DHA can be less than 0.1%, e.g., 0%, 0.01%, 0.02%, 0.03%, 0.04%, 0.05%, 0.06%, 0.07%, 0.08%, 0.09%, or less than 0.1%. In some embodiments, the crude algal oil is substantially free of DHA, e.g., 0% DHA by weight of total fatty acid content. In some instances, the crude oil that is substantially free of DHA contains no detectable amount of DHA, as determined by standard methods such as gas liquid chromatography and gas chromatography-mass spectrometry.

The crude algal oil can have a EPA:ARA ratio from about 5:1 to about 40:1, e.g., 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1 or 40:1. Alternatively, the EPA:ARA ratio can be from about 6:1 to about 30:1, e.g., 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1.

The fatty acids described herein can be found in the polar lipids and/or the neutral lipids of the crude algal oil. For instance, EPA can be present in lipids, such as monoacylglycerol (MAG), diacylglycerol (DAG), triacylglycerol (TAG), free fatty acid (FFA), phospholipid (PL), galactolipid (GL), phosphatidylcholine (PC), phosphatidylglycerol (PG), sulfoquinovosyldiacylglycerol (SQDG), diacylglycerol (DGDG), diacylglycerol trimethylhomoserine (DGTS), and monogalactosyldiacylglycerol (MGDG). ARA can found in lipids such as MAG, DAG, FFA, TAG, PE, SQDG, DGTS and MGDG.

In some embodiments, provided herein is a crude algal oil composition that includes a triacylglycerol fraction of at least about 2% to about 10%, e.g., at least about 2%, at least about 3%, at least about 4%, at least about 5%, at least about 6%, at least about 7%, at least about 8%, at least about 9% or at least about 10% by weight of the total fatty acid content. In some embodiments, the triacylglycerol fraction is at least about 2% by weight of the total fatty acid content. In some embodiments, about 3% to about 10% , e.g., about 3%, about 4%, about 5%, about 6%, about 7%, about 8%, about 9% or about 10%, of the eicosapentaenoic acid (EPA) of the crude algal oil is in the triacylglycerol fraction. In some embodiments, about 5% to about 15% , e.g., about 5%, about 6%, about 7%, about 8%, about 9%, about 10%, about 11%, about 12%, about 13%, about 14% or about 15% of the arachidonic acid (ARA) of the crude algal oil is in the triacylglycerol fraction. In some embodiments, there is substantially no docosahexaenoic acid (DHA) is in the triacylglycerol fraction of the crude algal oil. The crude algal oil described herein may be extracted from alga such as Nannochloropsis.

In some embodiments, provided herein is a crude algal oil composition that includes a monogalactosyl diacylglycerol (MGDG) fraction of at least about 5% by weight of the total fatty acid content. In some embodiments, about 30% to about 50%, e.g., about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49% or about 50% of the eicosapentaenoic acid (EPA) of the crude algal oil is in the MGDG fraction. In other embodiments, about 37% of the EPA of the crude algal oil is in the MGDG fraction. In some embodiments, about 10% to about 20%, e.g., about 10%, about 11%, about 12%, about 13%, abour 14%, about 15%, about 16%, about 17%, about 18%, about 19% or about 20% of the arachidonic acid (ARA) of the crude algal oil is in the MGDG fraction. In other embodiments, about 14% of the ARA of the crude algal oil is in the MGDG fraction. In some embodiments, substantially no docosahexaenoic acid (DHA), e.g., about 0% DHA, is in the MGDG fraction. The crude algal oil described herein may be extracted from alga such as Nannochloropsis.

In some embodiments, provided herein is a crude algal oil composition that includes a free fatty acid (FFA) fraction of at least about 1% by weight of the total fatty acid content, e.g., at least about 1%, 2%, 3%, 4%, 5%, or more by weight of the total fatty acid content. In some embodiments, about 5% to about 15%, e.g., about 5%, about 6%, about 7%, about 8%, about 9%, about 10%, about 11%, about 12%, about 13%, about 14% or about 15% of the EPA of the crude algal oil is in the FFA fraction. In other embodiments, about 11% of the EPA of the crude algal oil is in the FFA fraction. In some embodiments, about 15% to about 25%, e.g., about 15%, about 16%, about 17%, about 18%, about 19%, about 20%, about 21%, about 22%, about 23% about 24% or about 25% of the ARA of the crude algal oil is in the FFA fraction. In other embodiments, about 18% of the ARA of the crude algal oil is in the FFA fraction. In some embodiments, substantially no docosahexaenoic acid (DHA), e.g., about 0% DHA, is in the FFA fraction. The crude algal oil described herein may be extracted from alga such as Nannochloropsis. In some instance, the Nannochloropsis is a mutant strain, such as the T661 strain.

In some embodiments, provided herein is a crude algal oil composition that includes a phosphatidylcholine (PC) fraction of at least about 3% by weight of the total fatty acid content, e.g., at least about 3%, 4%, 5%, 6%, 7% or more by weight of the total fatty acid content. In some embodiments, about 5% to about 15%, e.g., about 5%, about 6%, about 7%, about 8%, about 9%, about 10%, about 11%, about 12%, about 13%, about 14% or about 15% of the EPA of the crude algal oil is in the PC fraction. In other embodiments, about 6% of the EPA of the crude algal oil is in the PC fraction. In some embodiments, about 10% to about 25%, e.g., about 10%, about 11%, about 12%, about 13%, about 14%, about 15%, about 16%, about 17%, about 18%, about 19%, about 20%, about 21%, about 22%, about 23% about 24% or about 25% of the ARA of the crude algal oil is in the PC fraction. In other embodiments, about 12% of the ARA of the crude algal oil is in the PC fraction. In some embodiments, substantially no docosahexaenoic acid (DHA), e.g., about 0% DHA, is in the PC fraction. The crude algal oil described herein may be extracted from alga such as Nannochlorophsis. In some instance, the Nannochlorophsis is a mutant strain, such as the T661 strain.

In some embodiments, one or more neutral lipid fractions of the crude algal oil, such as the monoacylglycerol fraction, the diacylglycerol fraction, the triacylglycerol fraction, or the free fatty acid fraction, comprise at least about 3%, at least about 4%, at least about 5%, at least about 6%, at least about 7%, at least about 8%, at least about 9%, at least about 10%, or at least about 11% of the EPA of the crude oil. In some embodiments, the free fatty acid fraction contains at least about 10% of the EPA of the crude oil. In some embodiments, the monoacylglycerol fraction contains at least about 3% of the EPA of the crude oil. In some embodiments, the diacylglycerol fraction contains at least about 4% of the EPA of the crude oil. In some embodiments, the triacylglycerol fraction contains at least about 7% of the EPA of the crude oil.

In some embodiments, one or more neutral lipid fractions of the crude algal oil, such as the monoacylglycerol fraction, the diacylglycerol fraction, the triacylglycerol fraction, or the free fatty acid fraction, comprise about 1% to about 20%, about 1% to about 15%, about 1% to about 10%, about 1% to about 5%, about 5% to about 20%, about 5% to about 15%, about 5% to about 10%, or about 10% to about 20% of the total EPA of the crude oil.

In some embodiments, the sterol ester, e.g., cholesterol ester, fraction of the crude oil contains no EPA.

In some embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, or monogalactosyldiacylglycerol fraction, comprises at least 1%, at least 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40% of the EPA of the crude oil.

In some embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, or monogalactosyldiacylglycerol fraction, comprise about 1% to about 40%, about 1% to about 35%, about 1% to about 30%, about 1% to about 20%, about 1% to about 10%, about 5% to about 40%, about 5% to about 35%, about 5% to about 30%, about 5% to about 25%, about 5% to about 20%, about 5% to about 15%, about 10% to about 40%, about 10% to about 35%, about 10% to about 25%, about 10% to about 20%, about 15% to about 40%, about 15% to about 35%, about 15% to about 30%, about 15% to about 25%, about 15% to about 20%, about 20% to about 40% of the EPA of the crude oil. In some embodiments, at least 35% or more of the EPA in the crude oil is located in monogalactosyl diacylglycerol lipids.

In other embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, monogalactosyldiacylglycerol fraction, comprise no EPA. For instance, EPA of the crude algal oil is not present in sterol esters, phosphatidylinositol lipids and/or phosphatidylserine lipids.

In some embodiments, one or more neutral lipid fractions of the crude algal oil, such as the monoacylglycerol fraction, the diacylglycerol fraction, the triacylglycerol fraction, or the free fatty acid fraction, comprise at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% of the total ARA of the crude oil. In some embodiments, the free fatty acid fraction contains at least about 16% of the ARA of the crude oil. In some embodiments, the monoacylglycerol fraction contains at least about 4% of the ARA of the crude oil. In some embodiments, the diacylglycerol fraction contains at least about 2% of the ARA of the crude oil. In some embodiments, the triacylglycerol fraction contains at least about 10% of the ARA of the crude oil.

In some embodiments, one or more neutral lipid fractions of the crude algal oil, such as the monoacylglycerol fraction, the diacylglycerol fraction, the triacylglycerol fraction, or the free fatty acid fraction, comprise about 1% to about 20%, about 1% to about 15%, about 1% to about 10%, about 1% to about 5%, about 5% to about 20%, about 5% to about 15%, about 5% to about 10%, or about 10% to about 20% of the total ARA of the crude oil.

In some embodiments, the sterol ester, e.g., cholesterol ester, fraction of the crude oil contains no ARA.

In some embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, or monogalactosyldiacylglycerol fraction, comprises at least 1%, at least 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20%, of the ARA of the crude oil. In some embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, or monogalactosyldiacylglycerol fraction, comprises no ARA.

In some embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, or monogalactosyldiacylglycerol fraction, comprise about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, or about 15% to about 20% of the total ARA of the crude oil.

In some embodiments, one or more polar lipid fractions of the crude algal oil, such as the phosphatidylcholine fraction, phosphatidylglycerol fraction, phosphatidylethanolamine fraction, phosphatidylinositol fraction, phosphatidylserine fraction, sulfoquinovosyl diacylglycerol fraction, digalactosyldiacylglycerol fraction, diacylglycerol trimethylhomoserine fraction, or monogalactosyldiacylglycerol fraction, comprise no ARA. In some instances, the fatty acid components of digalactosyl diacylglycerol lipids, phosphatidylinositol lipids and/or phosphatidylserine lipids are free of ARA.

The crude algal oil can be extracted from a freshly harvested algal biomass or can be extracted from a previously harvested biomass that has been stored under conditions that prevent spoilage. Known methods can be used to culture algae, to isolate an algal biomass from the culture, to extract a crude algal oil from the biomass, and to analyze the fatty acid profile of oils extracted from the biomass. Detailed descriptions are found in, e.g., Handayania et al., 2012, Scientific Reports, 1(2):180 (doi:10.4172/scientific reports.180) and Adarme-Vega et al., Microbial Cell Factories, 2012, 11:96, the contents of which is hereby incorporated by reference in the entirety for all purposes.

The crude algal oil can be derived from algae, including Chrysophyceae, Cryptophyceae, Prasinophyceae, Rhodophyceae, Xanthophyceae, Glaucophyceae, and Eustignatophyceae. In some embodiments, the algae is of the genus Nannochloropsis. In preferred embodiments, the Nannochloropsis is Nannochloropsis gaditana, Nannochloropsis granulate, Nannochloropsis limnetica, Nannochloropsis oceanica, Nannochloropsis oculata or Nannochloropsis salina.

In some embodiments, the crude algal oil is extracted from a chlorophyll deficient microalgae of the genus Nannochloropsis. The chlorophyll deficient algal cell can have a pale green phenotype as compared to a wild-type algal cell. In some embodiments, the chlorophyll deficient algal cell exhibits increased productivity and/or grow to a higher cell density as compared to a wild-type algal cell under the same lighting conditions. In particular, the chlorophyll deficient algal cell may exhibit robust growth under standard culture conditions. The chlorophyll deficient phenotype may be stable through multiple generations without selection.

The algal cells used to obtain the crude algal oil can be a wild-type algal cell or a mutant cell. In some embodiments, the chlorophyll deficient algal cell has one or more mutations as compared to a wild-type algal cell. In some instances, the chlorophyll deficient algal cell is a cell of the strain T661, or an isolate of a T661 culture. Detailed descriptions of an exemplary algal strain are found in, e.g., U.S. Provisional Application No. 61/800,029, filed Mar. 15, 2013 and U.S. Patent Application with attorney docket number 95844-902546 (titled “Chlorophyll Deficient Algal Cell with Improved Growth and Production” and filed Mar. 18, 2014), the contents of each are hereby incorporated by reference in their entirety for all purposes.

B. Culturing Algae

Algae can be cultured under conditions to promote the accumulation of algal oil high in EPA, low in ARA, and substantially free of DHA. For instance, the lipid content and compositions can be modulated by varying growth conditions such as light intensity, light-dark cycles, temperature, nutrient content, nutrient availability, salinity, pH, culture density, culture temperature, and other environmental conditions. Descriptions of growth conditions for Nannochloropsis are found in, e.g., Sukenik, A. “Chapter 3: Production of eicosapentaenoic acid by the marine Eustigmatophyte Nannochloropsis,” Chemicals from Microalgae., ed. Zvi Cohen, CRC Press, 1999, and Pal et al., Appl Microbiol Biotechnol, 2011, 90:1429-1441. Standard culture systems such as open ponds, e.g., open race way ponds, and photobioreactors can be used to grow algae.

To generate an algal biomass, standard methods, e.g., flocculation, centrifugation, and filtration (dead end filtration, microfiltration, ultrafiltration, pressure filtration, and tangential flow filtration) can be used for dewatering algae. For instance, cationic chemical flocculants, such as Al₂(SO₄)₃, FeCl₃, and Fe₂(SO₄)₃, can be used to coagulate harvested algae into a biomass.

The dried (dewatered) algal biomass may include at least about 10% lipids, e.g., about 10%, 20%, 30%, 40%, 50%, 60%, 70% or more lipid; at least about 15% carbohydrates, e.g., about 15%, 16%, 17%, 185, 19%, 20%, 21%, 22%, 23%, 24%, 25%, 26%, 27%, 28%, 29%, 30% or more carbohydrates; at least about 25% protein, e.g., about 25%, 26%, 27%, 28%, 29%, 20%, 21%, 32%, 33%, 34%, 35%, 36%, 37%, 38%, 39%, 40% or more protein; at least about 3% moisture, e.g., about 3%, 4%, 5%, 6%, 7%, 8%, 9%, 10%, 11%, 12%, 13%, 14%, 15%, 16%, 17%, 18%, 19%, 20% or more water; and at least about 5% ash, about 5%, 6%, 7%, 8%, 9%, 10%, 11%, 12%, 13%, 14%, 15%, 16%, 17%, 18%, 19%, 20% or more ash.

Algal cells or biomasses can be dried prior to use in obtaining the composition. Standard method of drying an algal biomass include freeze drying, air drying, spray drying, tunnel drying, vacuum drying (lyophilization), and a similar process. Alternatively, a harvested and washed biomass can be used directly produce the composition without drying. In some instances, the biomass is harvested and unwashed prior to performing the extraction method described herein. See, e.g., U.S. Pat. Nos. 5,130,242 and 6,812,009, the contents of which are herein incorporated by reference in their entirety.

C. Preparing Crude Algal Oil from the Algal Biomass

The algal oil of the present invention can be separated from the algal biomass by disruption methods that do not degrade the algal lipids. For instance, the algal cells of the biomass can be disrupted by, e.g., high-pressure homogenization, bead milling, expression/expeller press, sonication, ultrasonication, microwave irradiation, osmotic shock, electromagnetic pulsing, chemical lysis or grinding of dried algal biomass, to release the lipids and other intracellular components. Optionally, the oils can be separated from the algal cell debris by, e.g., centrifugation. For example, centrifugation produces an oil layer and an aqueous layer containing the cell debris.

Other useful methods for extracting fatty acids from algae include, but are not limited to: Bligh and Dyer's solvent extraction method; solvent extraction with a mixture of ionic liquids and methanol; hexane solvent extraction; ethanol solvent extraction; methanol solvent extraction; soxhlet extraction; supercritical fluid/CO₂ extraction; and organic solvent (e.g., benzene, cyclohexane, hexane, acetone, chloroform) extraction. See, e.g., Ratledge et al. “Chapter 13: Down-Stream Processing, Extraction, and Purification of Single Cell Oils,” Single Cell Oils, ed. Zvi Cohen and Colin Ratledge, AOCS Press, Champaign, Ill., 2005. The extraction method may affect the fatty acid composition recovered from the algal biomass. For instance, the concentration, volume, purity and type of fatty acid may be affected.

In some embodiments, ethanol is used to extract the crude algal oil from an algal biomass, such as dried algal biomass. It has been shown that ethanol extraction of lipids from algae can generate relatively high yields compared to other solvent-based methods.

After ethanol extraction, the extracted product can be further processed to remove water and polar components from the lipids. For example, as described in Fajardo et al., (Eur. J. Lipid Sci. Technol., 109 (2007) 120-126), the ethanol extract can be subjected to an apolar solvent extraction, e.g., hexane extraction to generate a biphasic extract containing a hexanic phase and a hydroalcoholic phase.

The extracted products may be processed using separation methods such as, but not limited to, distilling, decanting, and centrifuging. For example, the wet solids may be separated from the liquid fraction containing the crude algal oil. Alternatively, the ethanol extract can undergo an isolation step such as chromatography to produce a lipid-enriched composition.

D. Processing Crude Algal Oil into Fatty Acid Ethyl Esters

Crude algal oils provided herein can be used as starting material to more efficiently produce a product enriched in a fatty acid, such as EPA. For example, the algal oils of the invention can be subjected to various purification techniques known in the art, such as distillation or urea adduction, to produce a higher potency product with higher concentrations of EPA (e.g., about 60 to about 99% EPA, and more preferably about 90% to about 99% EPA). The crude oils can also be used in chemical reactions to produce compounds derived from fatty acids in the oils, such as esters and salts of EPA.

The crude algal oil described herein may be further processed to convert fatty acids present in the lipid fractions into free fatty acids (FFAs) by, for example, by saponification and neutralization. For instance, the FFAs can be extracted from the crude algal oil using a suitable solvent such as heptane.

Detailed descriptions for processing crude algal oil to fatty acid esters are found in, e.g., U.S. patent application Ser. No. 14/025,740, the disclosure of which is herein incorporated by reference in its entirety for all purposes. Briefly, free fatty acids can be mixed with an acid and ethanol in an esterification reaction to form ethyl esters. The esterification method forms a biphasic mixture comprising an aqueous layer and a solvent layer. In some embodiments, the reaction mixture is agitated and heated to a desired reaction temperature, such as about 50° C. to about 100° C. The esterification reaction is allowed to proceed until the desired percentage of conversion is achieved, such as for several minutes to about several hours. The aqueous phase can be removed prior to completion of the conversion. For instance, the reaction is allowed to proceed further after the aqueous layer is removed. This step can increase the conversion rate of free fatty acid to ethyl esters. After completion of the conversion, the fatty acid ethyl esters may be isolated by removing (separating) the aqueous layer, using standard methods known in the art such as decanting, centrifugation, and extraction.

The fatty acid ethyl esters may be processed to remove the C12 and C14 fatty acids, as well as any wax, thereby producing total algal oil. Exemplary total algal oil compositions are described in, e.g., U.S. patent application Ser. Nos. 14/025,762 and 14/025,756, the disclosures of which are herein incorporated by reference in their entirety for all purposes.

E. Producing EPA and POA Compositions

The crude algal oil and/or the total algal oil described herein can be can be further chemically or physically modified or processed by any known technique. In some embodiments, the method selected is based on the requirements of the end products, such as an omega-3 EPA composition comprising about 60% to about 99% EPA.

For instance, an EPA-enriched oil containing about 60% to about 99% (by dry weight of total fatty acid content) EPA can be isolated from the total algal oil by removing the C 16 fatty acids. In some embodiments, the algal oil is processed (refined) to produce an omega-3 EPA composition comprising about 60% to about 99% EPA, e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% EPA.

The EPA-enriched oil described herein can be blended with a POA-enriched composition, such as those described in, e.g., U.S. patent application Ser. No. 14/025,766, the contents of which are hereby incorporated by reference in the entirety for all purposes. For example, the omega-3/omega-7 oil blend composition can include 25% (by dry weight) of the EPA-enriched oil and 50% (by dry weight) of the POA-enriched composition; 35% of the EPA-enriched oil and 35% of the POA-enriched composition, and 50% of the EPA-enriched oil and 25% of the POA-enriched composition.

Exemplary algal fatty acid compositions, such as omega-3 and omega-7 blends are described in, e.g., U.S. patent application Ser. No. 14/025,762, the disclosures of which are herein incorporated by reference in their entirety for all purposes.

The omega-3 composition comprising about 60% to about 90% EPA, as described herein, can be used to produce various products, such as, but not limited to animal or fish feed, food additives, nutritional products, dietary products, cosmetics, industrial products, and pharmaceutical products. Descriptions of uses for these products are found in, e.g., U.S. patent application Ser. No. 14/025,772, the contents of which are herein incorporated by reference in its entirety for all purposes.

In some embodiments, the omega-3 composition is a food product. A food product is any food for non-human animal or human consumption, and includes both solid and liquid compositions. A food product can be an additive to animal or human foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods.

In some embodiments, the omega-3 composition is a non-human feed product. The term “non-human feed” or “non-human food” refers to any food intended for non-human animals, whether for fish; commercial fish; ornamental fish; fish larvae; bivalves; mollusks; crustaceans; shellfish; shrimp; larval shrimp; artemia; rotifers; brine shrimp; filter feeders; amphibians; reptiles; or mammals, such as dogs, cats, guinea pigs, rabbits, rats, mice, horses, monkeys, cows, cattle, pigs, sheep, and the like. An animal feed includes, but is not limited to, an aquaculture feed, a domestic animal feed including pet feed, a zoological animal feed, a work animal feed, a livestock feed, and combinations thereof.

In some embodiments, the omega-3 composition is a feed or feed supplement for any animal whose meat or products are consumed by humans, such as any animal from which meat, eggs, or milk is derived for human consumption. When fed to such animals, nutrients such as EPA can be incorporated into the flesh, milk, eggs or other products of such animals to increase their content of these nutrients.

In some embodiments, the omega-3 composition is a pharmaceutical composition. Suitable pharmaceutical compositions include, but are not limited to, an anti-inflammatory composition, a drug for treatment of coronary heart disease, a drug for treatment of arteriosclerosis, a chemotherapeutic agent, an active excipient, an osteoporosis drug, an anti-depressant, an anti-convulsant, an anti-Helicobacter pylori drug, a drug for treatment of neurodegenerative disease, a drug for treatment of degenerative liver disease, an antibiotic, a cholesterol lowering composition, and a triacylglycerol lowering composition. In some embodiments, the composition is a nutraceutical product. A nutraceutical product includes a food that is in a composition to be consumed or administered externally under the supervision of a physician and that is intended for the specific dietary management of a condition, for which distinctive nutritional requirements, based on recognized scientific principles, are established by medical evaluation.

For oral administration, the omega-3 composition can be combined with pharmaceutically acceptable carriers well known in the art. Such carriers enable the microbial oils of the invention to be formulated as tablets, pills, dragees, capsules, liquids, gels, syrups, slurries, suspensions and the like, for oral ingestion by a subject to be treated. In some embodiments, the dosage form is a tablet, pill or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinylpyrrolidone, agar, or alginic acid or a salt thereof such as sodium alginate. Pharmaceutical preparations that can be used orally include, but are not limited to, push-fit capsules made of gelatin, as well as soft, sealed capsules made of gelatin and a plasticizer, such as glycerol or sorbitol. In some embodiments, the dosage form is a vegetarian dosage form, in which the dosage form is not formed from and does not contain any components from an animal source. In some embodiments, the vegetarian dosage form is a vegetarian capsule.

In some embodiments, the omega-3 composition is a cosmetic. Cosmetics include, but are not limited to, emulsions, creams, lotions, masks, soaps, shampoos, washes, facial creams, conditioners, make-ups, bath agents, and dispersion liquids. Cosmetic agents can be medicinal or non-medicinal.

In some embodiments, the crude algal oil and/or the omega-3 composition are an industrial composition. In some embodiments, the composition is a starting material for one or more industrial products. An industrial product includes, but is not limited to, a polymer, a photographic photosensitive material, a detergent, an industrial oil, or an industrial detergent.

### IV. EXAMPLE

The following example is offered to illustrate, but not to limit, the claimed invention.

### Example 1

### Fatty Acid Profile of Crude Algal Oil

This example provides exemplary embodiments of the crude algal oil of the present invention. The crude oil was obtained from dried algae biomass by ethanol extraction.

Fatty acids were measured by transesterifying all free and ester-linked fatty acids to fatty acid methyl esters (FAMEs) in a solution of methanol and toluene, using hydrochloric acid as a catalyst. The FAMEs were extracted from the reaction mixture with hexanes, then concentrated and analyzed on an Agilent 6890 gas chromatograph equipped with a 30 m×0.25 mm×0.25 μm capillary column coated with a polyethylene glycol stationary phase (USP G16). Quantification was done relative to ethyl tricosanoate used as an internal standard. Fatty acid ethyl esters were measured using AOCS Official Method Ce 1b-89 (Fatty Acid Composition of Marine Oils by GLC).

A crude analytical lipid extract was made from biomass samples by extracting them with 2:1 methanol:chloroform (v:v) and washing the resulting extract with 1 molar potassium chloride, as described in Bligh & Dyer (Can J Biochem Physiol, 1959, 37:911-917). Two types of separations were done on silica thin-layer chromatographic plates. In the first separation, 1 milligram of the washed extract was separated using 80:20:2 hexanes:diethyl ether:formic acid (v:v:v), producing a polar lipid fraction (PL) along with fractions for monoacylglycerols (MAG), diacylglycerols (DAG), triacylglycerols (TAG), and a mixed steryl ester/hydrocarbon fraction (SE/HC). In the second separation, 1 milligram of the washed extract was separated in two dimensions. The first elution was done with 70:30:2.5 chloroform:methanol:water (v:v:v), then the plate was allowed to dry, rotated 90 degrees, and eluted again with 80:9:12:2.5 chloroform:methanol:acetic acid:water (v:v:v:v). Fractions identified by coelution with commercially available standards included phosphatidylinositol (PI), phosphatidylserine (PS), phosphatidylcholine (PC), phosphatidylglycerol (PG), digalactosyltrihomoserine (DGTS), digalactosyldiacylglycerol (DGDG), monogalactosyldiacylglycerol (MGDG), and sulfoquinovosyldiacylglycerol (SQDG). In both cases plates were visualized under 305 nm light after spraying with 0.05% primuline in 60:40 acetone:water (v:v). Quantification was done by transferring each spot into a tube and transesterifying lipids adsorbed to the silica directly to FAMEs, and quantifying FAMES as described above.

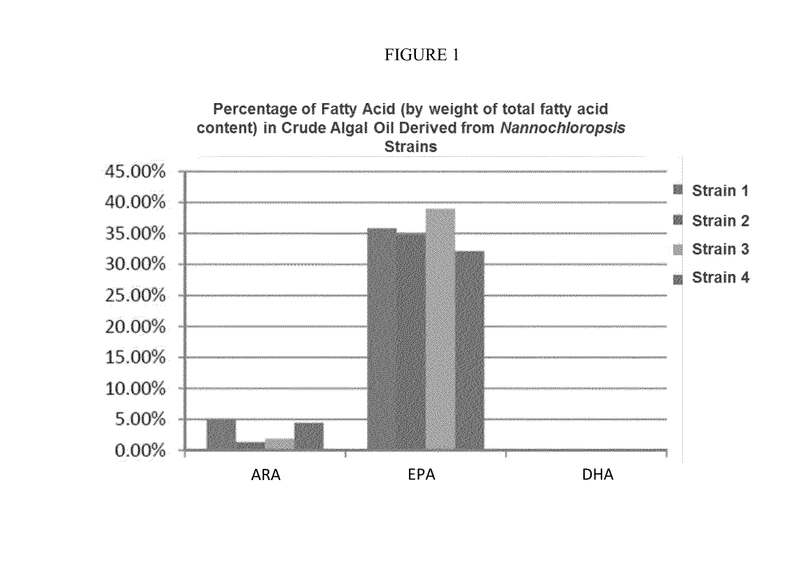

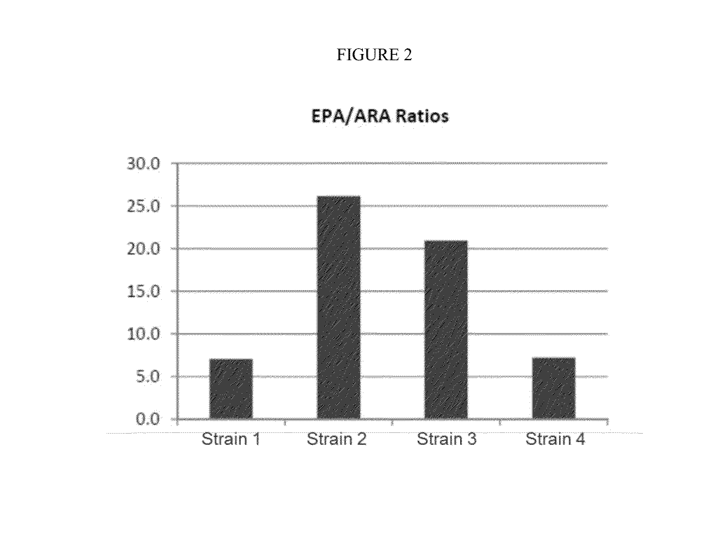

Crude algal oil was extracted from four Nannochloropsis strains of algae using the ethanol extraction method as described above. The strains produced crude algal oil containing less than 5% arachidonic acid (ARA; 20:4n6) by weight of total fatty acids (FIG. 1). The crude oil also contained about 31% to about 39% eicosapentaenoic acid (EPA; 20:5n3) and no docosahexaenoic acid (DHA; 22:6n3) (FIG. 1). Analysis of the EPA to ARA ratio also showed that the crude oils tested had a ratio ranging from about 6:1 to about 25:1 (FIG. 2). In particular, strain 1 had a EPA:ARA ratio of about 6:1; strain 2 had a ratio of about 26:1; strain 3 had a ratio of about 21:1 and strain 4 had an EPA:ARA ratio of about 6:1.

The fatty acid profiles of the whole biomass, crude algal oil, and refined oil (derived from the crude oil) are shown below in Table 1 as calculated as mg/g. Significantly higher amounts of EPA, as compared to ARA, were detected in all the samples (Table 1). No DHA or a significantly low amount of DHA was found in the whole biomass, crude algal oil and refined oil.

TABLE 1
Fatty Acid Compositions

| Fatty | Whole | Crude Algal | Refined |
|---|---|---|---|
| Acid | Biomass | Oil | Oil |
| (mg/g) | (ME) | (ME) | (ME) |
| 12:0 | 0.34 | 1.07 | 0.00 |
| 14:0 | 4.93 | 17.20 | 0.00 |
| 14:1n5 | 0.08 | 0.23 | |
| 16:0 | 19.34 | 69.19 | 4.08 |
| 16:1n7 | 19.99 | 73.97 | 3.01 |
| 16:2n4 | 0.19 | 0.84 | |
| 16:3n3 | 0.19 | 0.74 | |
| 18:0 | 0.63 | 2.45 | 4.40 |
| 18:1n7 | 0.57 | 2.30 | 4.65 |
| 18:1n9 | 2.71 | 10.23 | 19.30 |
| 18:2n6 | 1.30 | 5.32 | 11.01 |
| 18:3n3 | 0.54 | 3.64 | 2.55 |
| 18:3n6 | 0.30 | 1.18 | |
| 18:4n3 | 0.15 | 0.95 | |
| 20:0 | 0.10 | 0.29 | 2.09 |
| 20:1n9 | 0.11 | 0.42 | 2.85 |
| 20:2n6 | 0.01 | 0.45 | |
| 20:3n3 | 0.08 | 0.32 | |
| 20:3n6 | 0.31 | 1.17 | |
| 20:4n3 | 0.68 | 0.91 | |
| 20:4n6 | 4.09 | 14.50 | 118.56 |
| 20:5n3 | 16.15 | 61.21 | 632.88 |
| 22:0 | 0.24 | 0.31 | 4.44 |
| 22:1n9 | 0.02 | 0.01 | 0.56 |
| 22:6n3 | 0.05 | 0.00 | 0.28 |
| 24:1n9 | 0.00 | 0.09 | 0.00 |

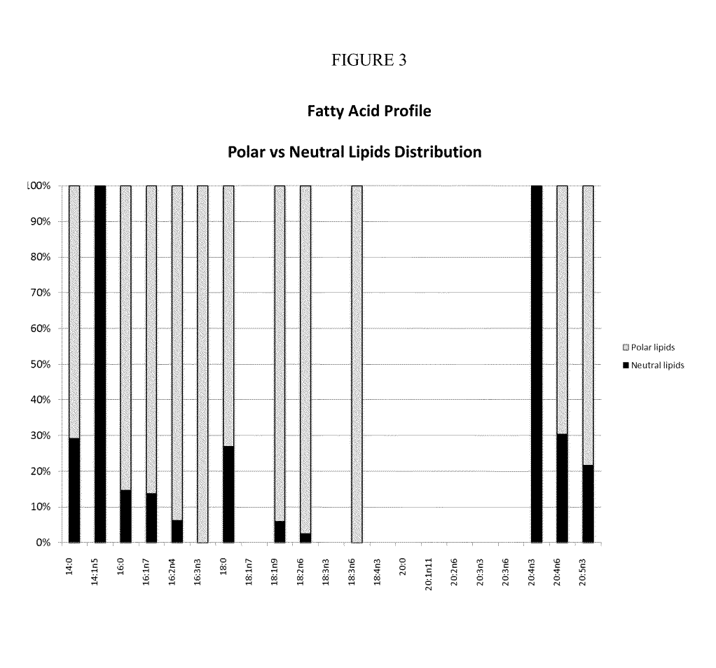

Analysis of the distribution of the fatty acids in the various lipid fractions showed that about 80% of the EPA of the crude algal oil was located in polar lipids and about 20% of the EPA in the crude oil was found in the neutral lipids (FIG. 3). For the ARA of the crude algal oil, about 33% of the total ARA was in the neutral lipids and about 67% was in the polar lipids (FIG. 3).

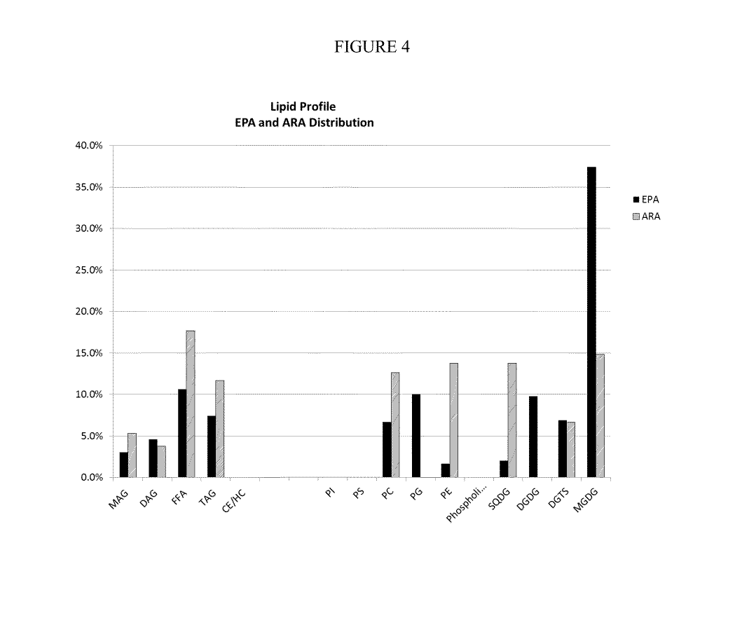

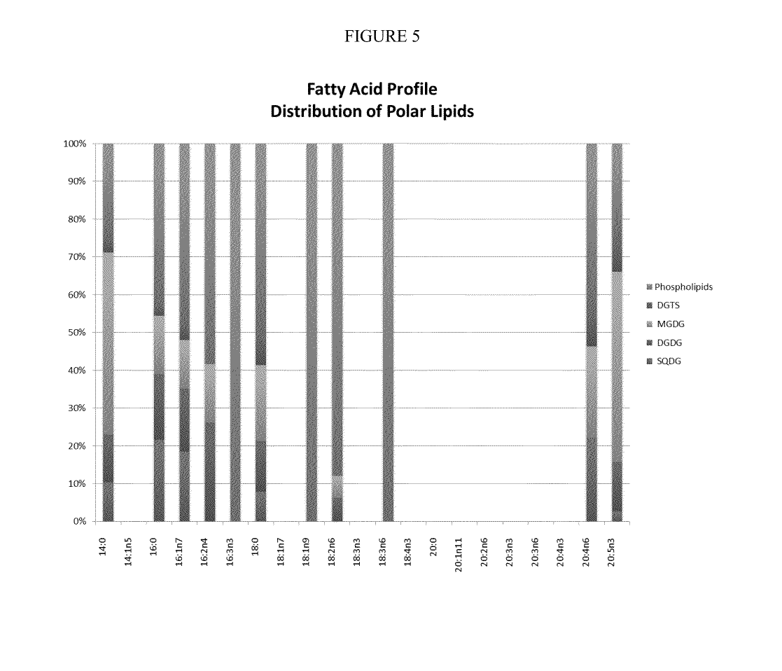

In an exemplary crude oil of the present invention, the distribution of EPA in the crude oil was as follows: the monoacylglycerol (MAG) fraction included about 3.0% of the EPA in the crude algal oil; the diacylglycerol (DAG) included about 4.6% of the EPA; the free fatty acid fraction (FFA) contained about 10.6% of the EPA; and the triacylglycerol (TAG) contained about 7.4% of the EPA in the crude oil. The EPA distribution in the polar lipids of the exemplary crude algal oil was as follows: 6.6% of the EPA in the crude oil was in the phosphatidylcholine (PC) fraction; 10.1% of the EPA was in the phosphatidylglycerol (PG) fraction; 1.7% of the EPA was in the phosphatidylethanolamine (PE); 2.0% of the EPA was in the sulfoquinovosyldiacylglycerol (SQDG) fraction; 9.7% of the EPA was in the digalactosyldiacylglycerol (DGDG) fraction; 6.9% was in the diacylglycerol trimethylhomoserine (DGTS) fraction; and 37.4% of the EPA was in the monogalactosyldiacylglycerol (MGDG) fraction. EPA was not detected in the cholesterol ester (CE/HC), phosphatidylinositol (PI), and phosphatidylserine (PS) fractions. See, FIG. 4. The distribution of EPA in the polar lipids of the crude algal oil is found in FIG. 5. Of the polar lipids, MGDG glycolipids contain the most EPA.

The distribution of ARA in the neutral lipids of the crude oil was as follows: the monoacylglycerol (MAG) fraction included about 5.3% of the ARA in the crude algal oil; the diacylglycerol (DAG) included about 3.7% of the ARA; the free fatty acid (FFA) fraction contained about 17.7% of the ARA; and the triacylglycerol (TAG) contained about 11.7% of the ARA in the crude oil. In the polar lipids of the exemplary crude algal oil, the ARA distribution was the following: 12.6% of the ARA in the crude oil was in the phosphatidylcholine (PC) fraction; 13.8% was in the phosphatidylethanolamine (PE); 13.7% of the ARA was in the sulfoquinovosyldiacylglycerol (SQDG) fraction; 6.6% was in the diacylglycerol trimethyl homoserine (DGTS) fraction; and 14.8% was in the monogalactosyl diacylglycerol (MGDG) fraction. No ARA was detected in the cholesterol ester (CE/HC), phosphatidylinositol (PI), phosphatidylserine (PS), phosphatidylglycerol (PG) and digalactosyl diacylglycerol (DGDG) fractions of the crude algal oil. See, FIG. 4.

Although the foregoing invention has been described in some detail by way of illustration and example for purposes of clarity of understanding, one of skill in the art will appreciate that certain changes and modifications may be practiced within the scope of the appended claims. In addition, each reference cited herein is incorporated by reference in its entirety to the same extent as if each reference was individually incorporated by reference.

1. A crude algal oil composition comprising
about 30% to about 35% eicosapentaenoic acid (EPA),
less than about 10% arachidonic acid (ARA.), and
less than about 0.5% docosahexaenoic acid (PHA) by weight of total fatty acids.

2. A crude algal oil composition comprising
a triacylglycerol fraction of at least about 2% by weight of total fatty acids,
wherein about 7% of the eicosapentaenoic acid (EPA) of the crude algal oil is in the triacylglycerol fraction,
wherein about 11% of the arachidonic acid (ARA) of the crude algal oil is in the triacylglycerol fraction, and
wherein substantially no docosahexaenoic acid (DHA) is in the triacylglycerol fraction.

3. A crude algal oil composition comprising
a monogalatosyldiacylglycerol (MGDG) fraction of at least about 5% by weight of total atty acids,
wherein about 37% of the eicosapentaenoic acid (EPA) of the crude algal oil is in the MGDG fraction,
wherein about 14% of the arachidonic acid (ARA) of the crude algal oil is in the MGDG fraction and,
wherein substantially no docosahexaenoic acid (DHA) is in the MGDG fraction.

4. The composition of claim 1, wherein DHA is less than about 0.1%.

5. The composition of claim 1, wherein the composition is substantially free of DHA.

6. The composition of claim 1, wherein the EPA:ARA ratio ranges from about 5:1 to about 40:1.

7. The composition of claim 1, wherein the EPA:ARA ratio ranges from about 6:1 to about 30:1.

8. The composition of claim 1, wherein about 80% of the EPA in the crude algal oil is in polar lipids and about 20% of the EPA is in neutral lipids.

9. The composition of claim 1, wherein about 37% of the EPA in the crude algal oil is in monogalactosyldiacylglycerol lipids.

10. The composition of claim 1, wherein about 10% of the EPA in the crude algal oil is in free fatty acids, phosphatidylglycerol lipids or digalactosyldiacylglycerol lipids.

11. The composition of claim 1 wherein the algal oil is derived from an algal biomass.

12. The composition of claim 1, wherein the algal biomass is a dried algal biomass.

13. The composition of claim 12, wherein the dried algal biomass comprises at least about 1 0% lipids, at least about 15% carbohydrates, at least about 25% protein, at least about 3% moisture, and at least about 1% ash.

14. The composition of claim 11, wherein the algal biomass comprises Nannochloropsis cells.

15. The composition of claim 14, wherein the Nannochloropsis is selected from the group consisting of Nannochloropsis gaditana, Nannochloropsis granulate, Nannochloropsis limnetica, Nannochloropsis oceanica, Nannochloropsis oculata and Nannochloropsis salina.

16. The composition of claim 14, wherein the Nannochloropsis is a mutant strain.

17. The composition of claim 1, wherein the crude algal oil is further processed to produce an omega-3 composition comprising about 60-99% EPA.

18. A method for producing the crude algal oil composition of claim 1, the method comprising:
(a) obtaining whole algal biomass;
(b) adding ethanol to the algal biomass; and
(c) extracting liquid from the algal biomass, thereby obtaining the crude algal oil.

19.-23. (canceled)

24. The method of claim 18, wherein the crude algal oil has less than about 0.1% DHA by weight of total fatty acids.

25. The method of claim 18, wherein the crude algal oil is substantially free of DHA.

26.-30. (canceled)
